(19) United States
(12) Patent Application Publication
(10) Pub. No.: US 2014/0370048 A1
(43) Pub. Date: Dec. 18, 2014

(54) COMPOSITION FOR TREATMENT OR PREVENTION OF HYPERLIPIDEMIA, CONTAINING ALCOHOL EXTRACTS OF OLIGOPORUS TEPHROLEUCUS

(71) Applicants: POSTECH ACADEMY-INDUSTRY FOUNDATION, Gyungbuk (KR); KYEONGSANGBUK-DO, Daegu (KR)

(72) Inventors: Sung Ho Ryu, Gyungbuk (KR); Hyeongji Lee, Busan (KR); Taehoon Lee, Seoul (KR); Jaeyoon Kim, Gyungbuk (KR); Jong-Vong Eun, Daegu (KR); Kang-Ha Kim, Gyungbuk (KR); Sang-Gap Shim, Gyungbuk (KR); Woo-Jae Cheon, Gyungbuk (KR)

(21) Appl. No.: 14/363,847

(22) PCT Filed: Dec. 7, 2012
(86) PCT No.: PCT/KR2012/010604

§ 371 Date: Jun. 9, 2014

(30) Foreign Application Priority Data
Dec. 9, 2011 (KR) 10-2011-0131850

(51) Int. Cl. A61K 36/07

(52) U.S. Cl. CPC A61K 36/07
USPC 424/195.15

(57) ABSTRACT

The present invention relates to a composition for the treatment or prevention of hyperlipidemia, the composition containing alcohol extracts of Oligoporus tephroleucus as an active ingredient, and to a medicine and a dietary supplement, containing the extracts.

### TECHNICAL FIELD

The present invention relates, in general, to an extract of Oligoporus tephroleucus, and, more particularly, to an alcohol extract of Oligoporus tephroleucus effective in treating and preventing hyperlipidemia, a use thereof, and a method of obtaining the same.

### BACKGROUND ART

Hyperlipidemia refers to an increased level of lipids in the blood, generally characterized by a condition when the total cholesterol level exceeds 240 mg/dL or triglyceride levels exceed 200 mg/dL. Hyperlipidemia is known as a main cause in the development of hypertension, myocardial infarction, strokes, etc., because it is apt to accompany the occlusion of blood vessels by cholesterol deposits in the blood leading to the obstruction of blood circulation. In Korea, there has been a steady increase in the number of hyperlipidemia patients. Thus various methods for the treatment and prevention of hyperlipidemia have been reported.

Hyperlipidemia may be usually due to genetic causes, like a metabolic disorder, on one hand, or may arise due to other underlying causes such as excessive caloric intake, a lack of exercise, obesity, on the other hand, either of which leads to cholesterol accumulation in the blood. (Bray G A, Popkin B M.: Dietary fat intake dose affect obesity. Am. J. Clin. Nutr 68: 1157-1173 (1998)). In particular, the main cause of hyperlipidemia appears to be the westernized high fat diet. Currently, research has been actively conducted globally to develop an effective material capable of inhibiting cholesterol absorption for preventing or treating hyperlipidemia. Accordingly, there is a growing interest in the anti-hyperlipidemia efficacy evaluation model. Meanwhile, triglycerides are also known to cause a decrease in the level of high-density lipoprotein (HDL) and allow low-density lipoprotein (LDL) to readily penetrate into the blood wall, thereby increasing the risk of developing cardiovascular diseases, strokes, and arteriosclerosis.

At present, statin-based drugs are commonly used as a treatment for hyperlipidemia. Statin-based drugs are Hmg-CoA reductase inhibitors, and are effective in preventing cholesterol synthesis and lowering LDL-cholesterol levels in the blood. In addition, ezetimibe, niacin, and fibrate formulations are also commonly used, but they have adverse side-effects such as muscular pains, constipation, digestive problems, and, in some rare cases, hepatic disorders.

Meanwhile, there are some 10,000 different mushrooms reported in the world. Due to their high value as edible foods and pharmaceutical uses, a considerable amount of research has been conducted in Europe, the U.S., Japan, etc., so as to secure them as beneficial microorganism resources. More specifically, according to research results, the physiologically active materials produced by mushrooms have few adverse effects and are thus safe in terms of toxicity. Additionally, they serve various functions, such as regulating functions within the human immune system, providing anti-cancer effects, regulation of metabolism, etc.

The inventors of the present invention, after prolonged research efforts into the development of natural substances having improved effects on hyperlipidemia by inhibiting cholesterol absorption in the body, have successfully discovered such substances not having any adverse effects in vivo, thus realizing the present invention.

### DISCLOSURE

### Technical Problem

The present invention aims to provide a composition effective for treating or preventing hyperlipidemia by inhibiting cholesterol and triglyceride absorption in the body without causing any adverse effects in the body, a pharmaceutical drug, and a functional health food containing the same.

### Technical Solution

In accordance with an aspect thereof, the present invention provides a composition containing a C₁-C₁₀ alcohol extract of Oligoporus tephroleucus as an active ingredient for treating or preventing hyperlipidemia.

Additionally, the present invention provides a pharmaceutical drug containing a C₁-C₁₀ alcohol extract of Oligoporus tephroleucus as an active ingredient for treating or preventing hyperlipidemia.

Furthermore, the present invention provides a method for treating or preventing obesity by administering a pharmaceutically effective amount of a C₁-C₁₀ alcohol extract of Oligoporus tephroleucus to a patient.

Still further, the present invention provides a functional health food containing a C₁-C₁₀ alcohol extract of Oligoporus tephroleucus as an active ingredient for improving or preventing hyperlipidemia.

In an exemplary embodiment of the present invention, there is provided a composition containing a C₁-C₁₀ alcohol extract of Oligoporus tephroleucus as an active ingredient for treating or preventing hyperlipidemia.

Oligoporus tephroleucus belongs to the Polyporaceae family, and is a tree brown rot fungi mushroom which grows in stumps of dead broadleaf tree from spring to autumn round. It has a white or yellow-orange color on the surface. Its piping is yellow and the shape of the piping is circle or amorphous. Its flesh is white color and very fragile, when it is dried.

While studying natural substances to replace statin-based drugs as an inhibitor to prevent cholesterol re-absorption in the small intestine or to reduce cholesterol levels in the blood, the inventors of the present invention discovered that an extract of Oligoporus tephroleucus, and in particular, a C₁-C₁₀ alcohol extract of Oligoporus tephroleucus, effectively inhibits cholesterol absorption in the blood. In addition, the C₁-C₁₀ alcohol extract of Oligoporus tephroleucus was also found to be very effective in inhibiting the absorption of triglycerides. The alcohol extract is not only effective in treating and preventing hyperlipidemia, but also effective in treating, preventing and improving obesity.

In particular, the alcohol extract of Oligoporus tephroleucus may be prepared according to a conventional method of plant extraction. In a preferable embodiment, Oligoporus tephroleucus, is dried and pulverized, followed by extraction by adding the resulting powder in an amount of 0.1 g to 20 g and preferably in an amount of 1 to 5 g to 100 mL of alcohol. When the amount of the pulverized resultant is too little relative to the volume of the extraction solvent, the effect of Oligoporus tephroleucus will not be sufficient and is thus undesirable. In contrast, when the amount of the pulverized resultant is in excess relative to the volume of the extraction solvent, there is no significant level of increase in the effect of Oligoporus tephroleucus, but results in an increase in production cost and is thus not desirable in terms of productivity.

In this regard, there is no particular limitation on the extraction conditions. Preferably, however, an alcohol extract of Oligoporus tephroleucus may be obtained by mixing Oligoporus tephroleucus with alcohol as an extraction solvent at a temperature of from 20 to 60° C. for from 12 to 36 hours, more preferably at from 30 to 40° C. for from 20 to 24 hours.

It takes a long time to extract the active ingredients at too low of a temperature. On the other hand, too high of an extraction temperature deteriorates the activities of the active ingredients. Furthermore, when the extraction is performed for too short of a time, an insufficient concentration of the active ingredients is obtained; whereas, when too long of an extraction time is undesirable in terms of productivity because the increment of the concentration of the extract is negligible compared to the extended time of extraction.

The alcohol extract of Oligoporus tephroleucus thus obtained may be filtrated using a filter cloth or the like, and after the centrifugation of the filtrate, the resulting pellet may be removed whereas the supernatant may be concentrated to dryness at reduced or normal pressure before lyophilization.

Although no particular limitation is imparted thereto, the alcohol extract of Oligoporus tephroleucus as an active ingredient is preferably in the range of from 0.001 to 50 wt % of the composition for treating or preventing hyperlipidemia according to an exemplary embodiment of the present invention. When the content of the alcohol extract of Oligoporus tephroleucus as an active ingredient is less than 0.001 wt %, the effect of inhibiting absorption of body cholesterol and triglycerides may be negligible; whereas a content exceeding 50 wt % is economically disadvantageous because a further increase in the content produces negligible effects. The composition more preferably contains the alcohol extract of Oligoporus tephroleucus in an amount of from 0.01 to 50 wt % and further more preferably in an amount of from 0.1 to 30 wt %.

In an exemplary embodiment of the present invention, there is provided a pharmaceutical drug containing an alcohol extract of Oligoporus tephroleucus as an active ingredient included in the composition. The pharmaceutical drug containing the alcohol extract of Oligoporus tephroleucus as an active ingredient may further include a carrier, an excipient, and diluents, as appropriate.

Examples of carriers, excipients, and diluents to be included in the pharmaceutical drug containing the alcohol extract of Oligoporus tephroleucus of the present invention as an active ingredient may include: lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and a mineral oil.

The pharmaceutical drug containing the alcohol extract of Oligoporus tephroleucus of the present invention as an active ingredient may be formulated into oral dosage forms such as powders, granules, tablets, suspensions, emulsions, syrups, etc., according to a conventional method, respectively.

The oral dosage formulations may include solid formulations and liquid formulations. Examples of the solid formulations for oral administration may include pills, powders, granules, capsules, etc. The solid formulations may include at least one excipient in addition to the extract, for example, starch, calcium carbonate, sucrose or lactose, gelatin, etc. In addition to the simple excipient, a lubricant such as magnesium stearate and talc may also be added. Examples of the liquid formulations for oral administration may include suspensions, liquid medicines for internal use, emulsions, syrups, etc. In addition to the simple diluent such as water and liquid paraffin, various excipients such as humectants, sweeteners, fragrants, and preservatives may also be used.

A preferred dosage of the composition of the present invention containing an alcohol extract of Oligoporus tephroleucus may vary depending on the individual subject's health conditions, body weight, severity of illness, formulation types, administration routes, etc., but may be appropriately chosen by one with ordinary skill in the art. Preferably, the daily dosage of the pharmaceutical drug of the present invention containing the alcohol extract of Oligoporus tephroleucus may be between 0.0001 to 100 mg/kg relative to the amount of the alcohol extract of Oligoporus tephroleucus, and more preferably, between 0.01 to 10 mg/kg. The pharmaceutical drug may be administered once or a few times daily. Nevertheless, the above dosage and the frequency of administration should not be construed as limiting the scope of the present invention in any manner.

In another exemplary embodiment of the present invention, there is provided a health functional food containing an alcohol extract of Oligoporus tephroleucus as an active ingredient for improving or preventing hyperlipidemia. The term “functional health food” used herein refers to a natural or processed product containing at least one nutrient, preferably one which has become readily edible by having undergone a certain level of processing.

Examples of the functional health food to add the alcohol extract of Oligoporus tephroleucus of the present invention may include various kinds of foods, beverages, gums, teas, vitamin complexes, etc. In addition, the foods of the present invention may include special nutrient foods (e.g., milk formulas, infants formulas, etc.), processed meat products, fish products, tofus, starch jellies, noodles (e.g., ramens, thin noodles, etc.), health supplement foods, seasoned foods (e.g., soy sauce, bean paste, red pepper paste, a mixed soy paste, etc.), sauces, confectioneries (e.g., snacks), milk products (e.g., fermented milks, cheeses, etc.), pickled foods (e.g., various kinds of kimchis, pickled vegetables, etc.), beverages (e.g., fruit juices, vegetable juices, soy milks, fermented beverages, etc.), and natural seasonings (e.g., ramen seasonings, etc.), but are not limited thereto. The above foods, beverages, or food additives may be manufactured according to a conventional manufacturing method.

The term, a functional food used herein, refers to a processed food designed to help regulate the body's natural biorhythms, prevent diseases or help a person recover from diseases, etc. Such foods are rendered with added value by means of physical, biochemical, and bioengineering technologies upon the group of foods or food compositions so that they can act and express the functions of a given food for a particular purpose, within live subjects. The functional food may further include a sitologically acceptable food supplement additive, and may further include other additives as appropriate, such as a carrier, an excipient, and a diluent, which are commonly used in the manufacture of functional foods.

The term, “beverage” used herein, is a collective term referring to all the drinks to be taken so as to quench thirst or enjoy the taste. The “beverage” should include as an active ingredient an alcohol extract of Oligoporus tephroleucus in a predetermined ratio but is not particularly limited regarding other ingredients. The beverage may further include various flavors or natural carbohydrates, etc., as is the case with conventional drinks. Examples of natural carbohydrates may include conventional sugars such as: monosaccharides, e.g., glucose, fructose, etc.; disaccharides, e.g., maltose, sucrose; and polysaccharides, e.g., dextrin, cyclodextrin, etc.; and a sugar alcohol such as xylitol, sorbitol, erythritol, etc. In addition to the sweeteners listed above, natural sweeteners such as thaumatin and stevia extracts (e.g., rebaudiocide A, glycyrrhizin, etc.), and other synthetic sweetenters (saccharin, aspartame, etc.) may be added. The natural carbohydrate may be added from about 1 to 20 g per 100 mL of a composition of the present invention, preferably from 5 to 12 g. Additionally, the composition of the present invention may further include the flesh of fruits for the manufacture of natural fruit juices, fruit juice beverages, and vegetable beverages.

Furthermore, the functional health food of the present invention may further include various nutrients, vitamins, minerals (electrolyte), natural and synthetic flavors, coloring agents, flavor enhancers (cheeses, chocolates, etc.), pectic acid and its salts, alginic acid and its salts, organic acids, protective colloid thickeners, pH adjusters, stabilizers, preservatives, glycerin, water, carbonating agents used in carbonated beverages, etc. The above ingredients may be used independently or in combination with others. Although the content of the additives may not be important, they may be used within the range of from 0 to 20 parts by weight per 100 parts by weight of the alcohol extract of Oligoporus tephroleucus.

The term, “functional beverage” used herein, refers to a processed beverage designed to help regulate the body's natural biorhythms, prevent diseases or help a person recover from diseases, etc. Such functional beverages are rendered with an added value by means of physical, biochemical, and bioengineering technologies on a group of beverages or beverage compositions so that they can act and express the functions of a given food for a particular purpose, within live subjects.

The functional beverage should include as an active ingredient an alcohol extract of Oligoporus tephroleucus in a predetermined ratio, but is not particularly limited regarding other ingredients. The functional beverage may further include various flavors or natural carbohydrates, etc., as is the case with conventional drinks. Examples of natural carbohydrates may include a conventional sugar such as monosaccharides, e.g., glucose, fructose, etc.; disaccharides, e.g., maltose, sucrose; and polysaccharides, e.g., dextrin, cyclodextrin, etc.; and a sugar alcohol such as xylitol, sorbitol, erythritol, etc. In addition to the sweeteners above, natural sweeteners, such as thaumatin and stevia extracts (e.g., rebaudiocide A, glycyrrhizin, etc.), and synthetic flavors (saccharin, aspartame, etc.) may be added. The natural carbohydrate may be added from about 1 to 20 g per 100 mL of a composition of the present invention, preferably from 5 to 12 g. Additionally, the composition of the present invention may further include flesh of fruits for the manufacture of natural fruit juices, fruit juice beverages, and vegetable beverages.

Additionally, the functional health food for improving or preventing hyperlipidemia may contain the alcohol extract from 0.01 to 15 wt % of the total food of the functional health food, and the beverage composition may contain the alcohol extract from 0.02 to 5 g per 100 mL, preferably from 0.3 to 1 g.

In an exemplary embodiment of the present invention, there is provided a method for manufacturing the alcohol extract of Oligoporus tephroleucus.

The method for manufacturing the water extract of Oligoporus tephroleucus may include preparing Oligoporus tephroleucus, mixing Oligoporus tephroleucus with alcohol, and extracting the resulting mixture at a temperature of from 20 to 60° C. for from 12 to 36 hours. The details of the time, temperature, and mixed ratio of a solvent for the extraction of Oligoporus tephroleucus are the same as described above.

### Advantageous Effects

According to the present invention, the composition including an alcohol extract of Oligoporus tephroleucus as an active ingredient can inhibit the absorption of cholesterol and triglycerides in the body, and is thus useful for treating or preventing hyperlipidemia.

### DESCRIPTION OF DRAWINGS

FIG. 1 is a graph showing the comparative results of the inhibitory effect between the extracts of Oligoporus tephroleucus prepared in Example and Comparative Example upon CEL activity.

FIG. 2 is a graph showing the results of the amount of cholesterol absorbed into blood from the digestive tract when the alcohol extract of Oligoporus tephroleucus prepared according to an exemplary embodiment of the present invention, and orlistat, a conventional fat inhibitor, were compared.

### MODE FOR INVENTION

A better understanding of the present invention regarding its invention features and effects may be obtained through the following examples which are set forth to illustrate, but are not to be construed as the limit of the present invention. The details of the present invention are described infra.

### Example

### Preparation of an Ethanol Extract of Oligoporus tephroleucus

A mixture of 5 g of a dry powder of Oligoporus tephroleucus in 100 mL of ethanol (99.9% (v/v), as a solvent), was subjected to extraction at 37° C. for 24 hours in a shaking incubator. After centrifugation at 2500 rpm for 10 minutes, the supernatant was recovered as an alcohol extract of Oligoporus tephroleucus.

### Comparative Example

### Preparation of a Water Extract of Oligoporus tephroleucus

A water extract of Oligoporus tephroleucus was prepared in the same manner as in Example except that 100 ml of water was used as an extraction solvent instead of ethanol.

### Experimental Example

### 1. Measurement of the Activity of Pancreatic Cholesterol Esterase Upon Treatment with Oligoporus tephroleucus Extracts

Pancreatic cholesterol esterase is known to non-specifically recognize substrates having an acyl chain and separate the acyl chain therefrom. For use in measuring the effect of the Oligoporus tephroleucus extracts prepared in the Example and the Comparative Example on the activity thereof, An Enzyme, porcine pancreatic cholesterol esterase was purchased from Sigma Chemical.

The change in the activity of pancreatic lipase was observed by a color reaction considering that pancreatic lipase can decompose p-nirophenylbutyrate, a chromogenic substrate. The inhibitory effects of the extracts upon the enzyme were measured by a microplate reader based on the change in absorbance at 405 nm, and the optical density of a group not treated with the extracts was calculated in % with reference to the absorbance at 405 nm as a base value.

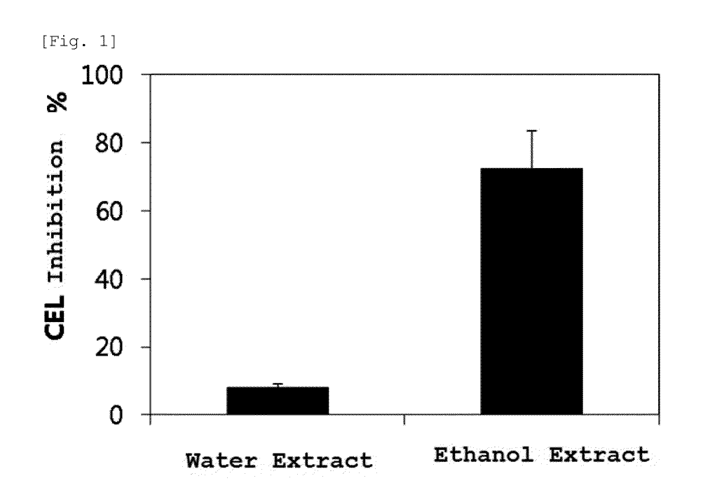

The changes in the enzyme activity by the treatment with extracts prepared in the Example and the Comparative Example were measured. The results revealed that the ethanol extract of Oligoporus tephroleucus prepared in the Example considerably decreased the activity of pancreatic lipase, whereas the water extract of Oligoporus tephroleucus prepared in the Comparative Example showed almost no inhibitory effect against the activity of pancreatic lipase. The results are briefly shown in FIG. 1.

### 3. Measurement of the Amount of Cholesterol Absorption Upon Treatment with Ethanol Extracts of Oligoporus tephroleucus

In order to confirm whether the ethanol extract of Oligoporus tephroleucus prepared in the Example, which exhibited an inhibitory effect upon the pancreatic cholesterol esterase, also shows any inhibitory effect at the level of an individual subject, the cholesterol absorption in the digestive tract was measured.

Cholesterol oleate (Amersham, 100 μCi/mL) radiolabeled with ³H was mixed with 1% (w/v) sodium carboxylmethyl cellulose (CMC-Na), 1% (v/v) Tween 80, and a non-labeled cholesterol oleate, and then 0.1 mL of the mixture was forcibly administered into each mouse through an oral zonde so that 1 μCi could be delivered to each mouse.

Here, the mouse in the control group (an 8-week old male Balb/c with body weight of 25 g) was treated with 400 μg of Orlistat (20 mg/kg), which has an inhibitory effect against cholesterol absorption, and the mouse in the experimental group (an 8-week old male Balb/c with body weight of 25 g) was treated with 20 ml of the ethanol extract (10 mg/mL) of Oligoporus tephroleucus prepared in the Example after concentrating it into 0.1 mL.

Six hours after the administration, blood samples were collected from the mice, and plasma was obtained by centrifuging the blood at 40° C. at the rate of 14000 rpm, and the radioactive dose was measured by a liquid scintillator (Beta counter, Beckman LS1801). The Control group was not treated with anything that may inhibit the absorption of cholesterol. The amount of cholesterol absorption was determined as a relative value by dividing the radioactive dose of each group into the radioactive dose of the control group, in which the radioactive dose of the control group was set at ‘1’.

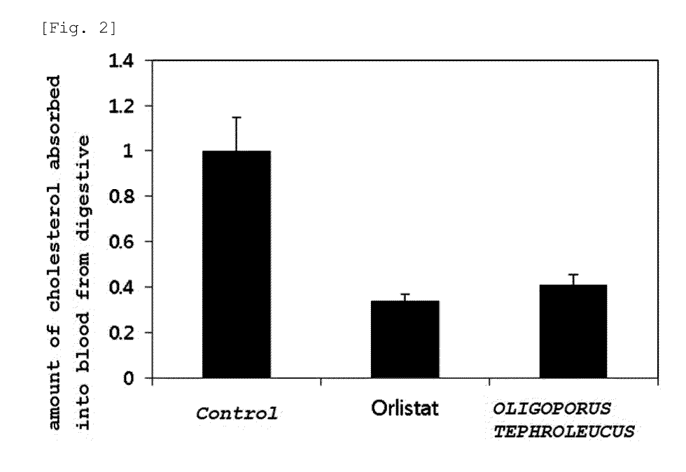

The ethanol extract of Oligoporus tephroleucus of the present invention showed a significant inhibitory effect upon the cholesterol absorption, as in the control group treated with Orlistat. The results are shown in FIG. 2. In particular, the result on the control group in FIG. 2 was obtained from the blood sample of a mouse not treated with any cholesterol absorption inhibitor.

Examples of formulations manufactured using the alcohol extract of Oligoporus tephroleucus are described herein below, but the formulations of the present invention are not limited thereto.

### Manufacturing Example

### Formulations Manufactured Using Ethanol Extract of Oligoporus tephroleucus

### Manufacturing Example 1

### Powders

| | |
|---|---|
| powdered ethanol extract of Oligoporus tephroleucus | 20 mg |
| lactose | 100 mg |
| talc | 10 mg |

The above ingredients were mixed and loaded to a sealed pouch to obtain the formulation in the form of powders.

### Manufacturing Example 2

### Tablets

| | |
|---|---|
| powdered ethanol extract of Oligoporus tephroleucus | 10 mg |
| corn starch | 100 mg |
| lactose | 100 mg |
| magnesium stearate | 2 mg |

The above ingredients were mixed and tableted according to a conventional tableting method to obtain the formulation in the form of a tablet.

### Manufacturing Example 3

### Capsules

| | |
|---|---|
| powdered ethanol extract of Oligoporus tephroleucus | 10 mg |
| crystalline cellulose | 3 mg |
| lactose | 14.8 mg |
| magnesium stearate | 0.2 mg |

The above ingredients were mixed and filled into a gelatin capsule according to a conventional capsule manufacturing method to obtain the formulation in the form of a capsule.

### Manufacturing Example 4

### Liquid

| | | |
|---|---|---|
| powdered ethanol extract of Oligoporus tephroleucus | 20 | mg |
| isomerose | 10 | g |
| mannitol | 5 | g |

| | |
|---|---|
| purified water | adequate |

According to a conventional liquid manufacturing method, each of the above ingredients was dissolved by adding purified water, added with an appropriate amount of a lemon flavor, mixed together, added with purified water to a final volume of 100 mL, filled into a brown bottle, and then sterilized to obtain the formulation in the form of a liquid.

### Manufacturing Example 5

### Health Foods

| powdered ethanol extract of Oligoporus tephroleucus | 20 | mg |
|---|---|---|
| vitamin mixture | adequate | |
| vitamin A acetate | 70 | μg |
| vitamin E | 1.0 | mg |
| vitamin B1 | 0.13 | mg |
| vitamin B2 | 0.15 | mg |
| vitamin B6 | 0.5 | mg |
| vitamin B12 | 0.2 | μg |
| vitamin C | 10 | mg |
| biotin | 10 | μg |
| nicotinic acid amide | 1.7 | mg |
| folic acid | 50 | μg |
| calcium pantothenate | 0.5 | mg |
| mixture of minerals | adequate | |
| ferrous sulfate | 1.75 | mg |
| zinc oxide | 0.82 | mg |
| magnesium carbonate | 25.3 | mg |
| potassium phosphate, monobasic | 15 | mg |
| calcium phosphate, dibasic | 55 | mg |
| potassium citrate | 90 | mg |
| calcium carbonate | 100 | mg |
| magnesium chloride | 24.8 | mg |

Although the mixed ratios of the vitamins and the mixture of minerals disclosed above preferred ones composed of ingredients relatively suitable for manufacturing health foods, various modifications or changes in the mixing ratios may be possible. The above ingredients may be mixed according to the conventional method of manufacturing health foods to be manufactured in the form of granules, and used for manufacturing health food compositions according to the conventional methods.

### INDUSTRIAL APPLICABILITY

Although the preferred embodiment(s) of the present invention have(has) been disclosed for illustrative purposes, those skilled in the art will appreciate that various modifications, additions and substitutions are possible, without departing from the scope and spirit of the invention as disclosed in the accompanying claims.

1. A composition comprising a C₁-C₁₀ alcohol extract of Oligoporus tephroleucus as an active ingredient for treating or preventing hyperlipidemia.

2. The composition of claim 1, wherein the composition comprises a C₁-C₅ alcohol extract.

3. The composition of claim 1, wherein the composition comprises an ethanol extract.

4. The composition of claim 3, wherein the ethanol is 90˜99.9% (v/v) ethanol.

5. The composition of claim 1, wherein the extract of Oligoporus tephroleucus is obtained by extracting Oligoporus tephroleucus with 90˜99.9% (v/v) ethanol at a temperature of from 20 to 60° C. for from 12 to 36 hours.

6. The composition of claim 1, wherein the extract of Oligoporus tephroleucus is obtained using from 0.1 to 20 g of Oligoporus tephroleucus per 100 mL of C₁-C₁₀ alcohol.

7. The composition of claim 1, wherein the C₁-C₁₀ alcohol extract of Oligoporus tephroleucus is from 0.001 to 50 wt %.

8. The composition of claim 1, wherein the extract of Oligoporus tephroleucus inhibits the absorption of cholesterol.

9. The composition of claim 1, wherein the extract of Oligoporus tephroleucus deteriorates the activity of pancreatic cholesterol esterase.

10. A functional health food comprising C₁-C₁₀ alcohol extract of Oligoporus tephroleucus as an active ingredient for improving or preventing hyperlipidemia.

11. The functional health food of claim 10, wherein the functional health food is a food, a food additive or a drink.

12. The functional health food of claim 10, wherein the functional health food inhibits the absorption of cholesterol.

13. An alcohol extract of Oligoporus tephroleucus, which inhibits the absorption of cholesterol in the blood.

14. The alcohol extract of Oligoporus tephroleucus of claim 13, which deteriorates the activity of pancreatic cholesterol esterase.

15. The alcohol extract of Oligoporus tephroleucus of claim 13, wherein the alcohol extract of Oligoporus tephroleucus is obtained by extracting Oligoporus tephroleucus with 90˜99.9% (v/v) ethanol at a temperature of from 20 to 60° C. for from 12 to 36 hours.
